(19) United States
(12) Patent Application Publication
(10) Pub. No.: US 2019/0263419 A1
(43) Pub. Date: Aug. 29, 2019

(54) AUTONOMOUS VEHICLE CONTROL BY COMPARATIVE TRANSITION PREDICTION

(71) Applicant: Ford Motor Company, Dearborn, MI (US)

(72) Inventors: Kwaku O. PRAKAH-ASANTE, Commerce Township, MI (US); Gary Steven STRUMOLO, Canton, MI (US); Reates CURRY, Ann Arbor, MI (US)

(21) Appl. No.: 16/348,906

(22) PCT Filed: Nov. 14, 2016
(86) PCT No.: PCT/US2016/061745

§ 371 Date: May 10, 2019

(51) Int. Cl. B60W 40/09; B60K 28/06

(52) U.S. Cl. CPC B60W 40/09; B60W 2040/0872; B60K 28/06

(57) ABSTRACT

Vehicles can be equipped to operate in both autonomous and occupant piloted mode. Vehicles can monitor physiological signals and determine when an occupant is in a transition state thereby predicting an inattentive, sleepy state. When a transition state is determined the occupant can be alerted and the vehicle can be piloted autonomously for some period of time.

### BACKGROUND

Vehicles can be equipped to operate in both autonomous and occupant piloted mode. Vehicles can be equipped with computing devices, networks, sensors and controllers to pilot the vehicle and to assist an occupant in piloting the vehicle. Even when a vehicle is operated autonomously, it may be important for a vehicle occupant to supervise and be ready and able to assume control of the vehicle.

### BRIEF DESCRIPTION OF THE DRAWINGS

FIG. 1 is a block diagram of an example vehicle.

FIG. 2 is a diagram of an example comparative transition prediction system.

FIG. 3 is a diagram of example physiological signals.

FIG. 4 is a diagram of second example physiological signals.

FIG. 5 is a diagram of example transitional engagement values.

FIG. 6 is a diagram of second example transitional engagement values.

FIG. 7 is a flowchart diagram of a process to pilot a vehicle based comparative transition prediction.

FIG. 8 is a flowchart diagram of a process to output transition state aᵢ.

### DETAILED DESCRIPTION

Vehicles can be equipped to operate in both autonomous and occupant piloted mode. By a semi- or fully-autonomous mode, we mean a mode of operation wherein a vehicle can be piloted by a computing device as part of a vehicle information system having sensors and controllers. The vehicle can be occupied or unoccupied, but in either case the vehicle can be piloted without assistance of an occupant. For purposes of this disclosure, an autonomous mode is defined as one in which each of vehicle propulsion (e.g., via a powertrain including an internal combustion engine and/or electric motor), braking, and steering are controlled by one or more vehicle computers; in a semi-autonomous mode the vehicle computer(s) control(s) one or two of vehicle propulsion, braking, and steering.

Vehicles can be equipped with computing devices, networks, sensors and controllers to pilot the vehicle and to determine maps of the surrounding real world including features such as roads. Vehicles can be piloted and maps can be determined based on locating and identifying road signs in the surrounding real world. By piloting we mean directing the movements of a vehicle so as to move the vehicle along a roadway or other portion of a path.

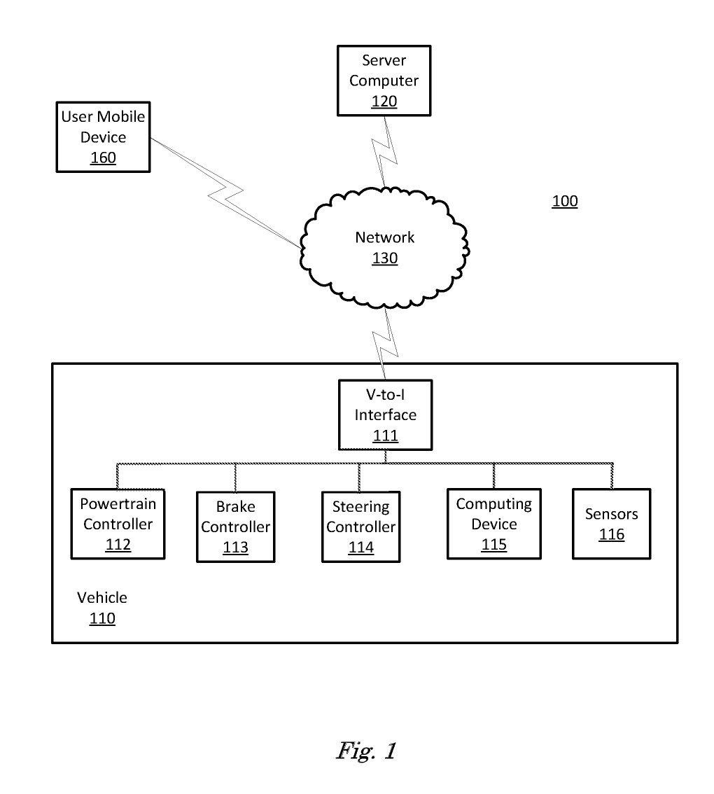

FIG. 1 is a diagram of a vehicle information system 100 that includes a vehicle 110 operable in autonomous (“autonomous” by itself in this disclosure means “fully autonomous”) and occupant piloted (also referred to as non-autonomous) mode in accordance with disclosed implementations. Vehicle 110 also includes one or more computing devices 115 for performing computations for piloting the vehicle 110 during autonomous operation. Computing devices 115 can receive information regarding the operation of the vehicle from sensors 116.

The computing device 115 includes a processor and a memory such as are known. Further, the memory includes one or more forms of computer-readable media, and stores instructions executable by the processor for performing various operations, including as disclosed herein. For example, the computing device 115 may include programming to operate one or more of vehicle brakes, propulsion (e.g., control of acceleration in the vehicle 110 by controlling one or more of an internal combustion engine, electric motor, hybrid engine, etc.), steering, climate control, interior and/or exterior lights, etc., as well as to determine whether and when the computing device 115, as opposed to a human operator, is to control such operations.

The computing device 115 may include or be communicatively coupled to, e.g., via a vehicle communications bus as described further below, more than one computing devices, e.g., controllers or the like included in the vehicle 110 for monitoring and/or controlling various vehicle components, e.g., a powertrain controller 112, a brake controller 113, a steering controller 114, etc. The computing device 115 is generally arranged for communications on a vehicle communication network such as a bus in the vehicle 110 such as a controller area network (CAN) or the like; the vehicle 110 network can include wired or wireless communication mechanism such as are known, e.g., Ethernet or other communication protocols.

Via the vehicle network, the computing device 115 may transmit messages to various devices in the vehicle and/or receive messages from the various devices, e.g., controllers, actuators, sensors, etc., including sensors 116. Alternatively, or additionally, in cases where the computing device 115 actually comprises multiple devices, the vehicle communication network may be used for communications between devices represented as the computing device 115 in this disclosure. Further, as mentioned below, various controllers or sensing elements may provide data to the computing device 115 via the vehicle communication network.

In addition, the computing device 115 may be configured for communicating through a vehicle-to-infrastructure (V-to-I) interface 111 with a remote server computer 120, e.g., a cloud server, via a network 130, which, as described below, may utilize various wired and/or wireless networking technologies, e.g., cellular, BLUETOOTH® and wired and/or wireless packet networks. The computing device 115 also includes nonvolatile memory such as is known. Computing device 115 can log information by storing the information in nonvolatile memory for later retrieval and transmittal via the vehicle communication network and a vehicle to infrastructure (V-to-I) interface 111 to a server computer 120 or user mobile device 160.

As already mentioned, generally included in instructions stored in the memory and executed by the processor of the computing device 115 is programming for operating one or more vehicle 110 components, e.g., braking, steering, propulsion, etc., without intervention of a human operator. Using data received in the computing device 115, e.g., the sensor data from the sensors 116, the server computer 120, etc., the computing device 115 may make various determinations and/or control various vehicle 110 components and/or operations without a driver to operate the vehicle 110. For example, the computing device 115 may include programming to regulate vehicle 110 operational behaviors such as speed, acceleration, deceleration, steering, etc., as well as tactical behaviors such as a distance between vehicles and/or amount of time between vehicles, lane-change, minimum gap between vehicles, left-turn-across-path minimum, time-to-arrival at a particular location and intersection (without signal) minimum time-to-arrival to cross the intersection.

Controllers, as that term is used herein, include computing devices that typically are programmed to control a specific vehicle subsystem. Examples include a powertrain controller 112, a brake controller 113, and a steering controller 114. A controller may be an electronic control unit (ECU) such as is known, possibly including additional programming as described herein. The controllers may communicatively be connected to and receive instructions from the computing device 115 to actuate the subsystem according to the instructions. For example, the brake controller 113 may receive instructions from the computing device 115 to operate the brakes of the vehicle 110.

The one or more controllers 112, 113, 114 for the vehicle 110 may include known electronic control units (ECUs) or the like including, as non-limiting examples, one or more powertrain controllers 112, one or more brake controllers 113 and one or more steering controllers 114. Each of the controllers 112, 113, 114 may include respective processors and memories and one or more actuators. The controllers 112, 113, 114 may be programmed and connected to a vehicle 110 communications bus, such as a controller area network (CAN) bus or local interconnect network (LIN) bus, to receive instructions from the computer 115 and control actuators based on the instructions.

Sensors 116 may include a variety of devices known to provide data via the vehicle communications bus. For example, a radar fixed to a front bumper (not shown) of the vehicle 110 may provide a distance from the vehicle 110 to a next vehicle in front of the vehicle 110, or a global positioning system (GPS) sensor disposed in the vehicle 110 may provide geographical coordinates of the vehicle 110. The distance provided by the radar or the geographical coordinates provided by the GPS sensor may be used by the computing device 115 to operate the vehicle 110 autonomously or semi-autonomously.

The vehicle 110 is generally a land-based autonomous vehicle 110 having three or more wheels, e.g., a passenger car, light truck, etc. The vehicle 110 includes one or more sensors 116, the V-to-I interface 111, the computing device 115 and one or more controllers 112, 113, 114.

The sensors 116 may be programmed to collect data related to the vehicle 110 and the environment in which the vehicle 110 is operating. By way of example, and not limitation, sensors 116 may include, e.g., altimeters, cameras, LIDAR, radar, ultrasonic sensors, infrared sensors, pressure sensors, accelerometers, gyroscopes, temperature sensors, pressure sensors, hall sensors, optical sensors, voltage sensors, current sensors, mechanical sensors such as switches, etc. The sensors 116 may be used to sense the environment in which the vehicle 110 is operating such as weather conditions, the grade of a road, the location of a road or locations of neighboring vehicles 110. The sensors 116 may further be used to collect dynamic vehicle 110 data related to operations of the vehicle 110 such as velocity, yaw rate, steering angle, engine speed, brake pressure, oil pressure, the power level applied to controllers 112, 113, 114 in the vehicle 110, connectivity between components and electrical and logical health of the vehicle 110.

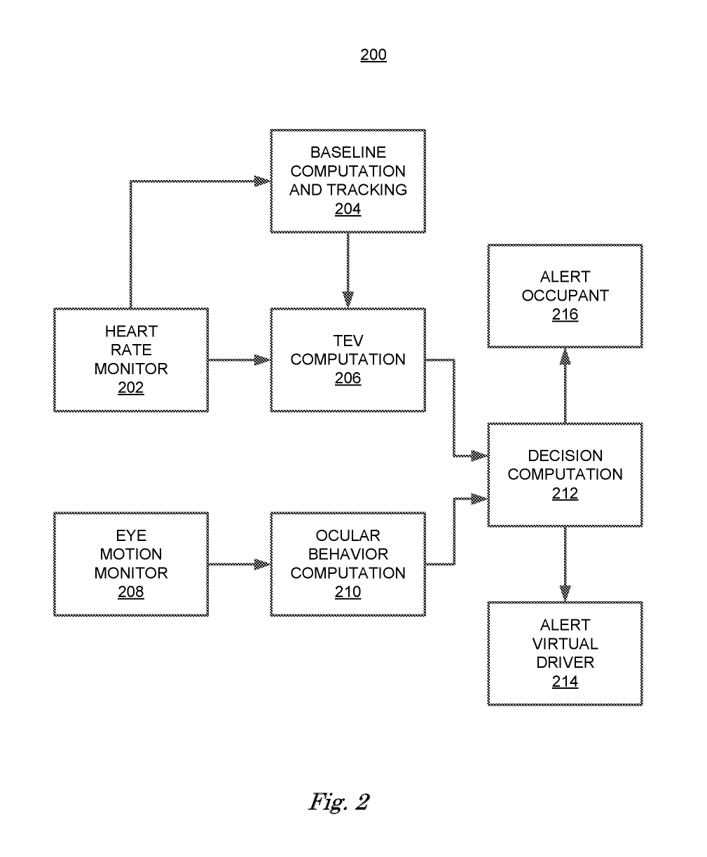

FIG. 2 is a diagram of a comparative transition prediction system 200. Comparative transition prediction system 200 can be implemented as one or more combinations of hardware and software programs executing on computing device 115 included in vehicle 110, for example. Comparative transition prediction system 200 can include a heart rate monitor 202. Heart rate monitor 202 can acquire heart rate data from vehicle 110 occupant. Acquire means to receive, obtain, measure, gauge, read, or in any manner whatsoever acquire. Heart rate monitor 202 can include wearable devices including watches, wrist bands, fobs, pendants or articles of clothing that can detect a wearer's heart rate and transmit it to computing device 115, for example. Heart rate monitor 202 can also include non-contact devices such as infrared video sensors or microphones that can detect an occupant's heart rate by optical or audio means, for example.

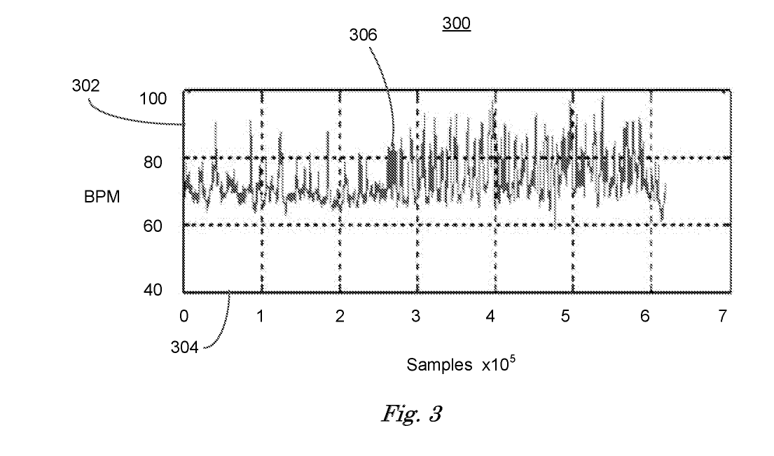

Heart rate monitor 202 can acquire heart rate data 300 as shown in FIG. 3. FIG. 3 is a graph of example heart rate data 300 from heart rate monitor 202 that graphs heart rate in beats per minute (BPM) on the Y-Axis 302 vs. number of samples×10⁵ on the X-Axis 304. Heart rate data can be sampled many times per second, for example, to create a heart rate data curve 306. The intervals on X-Axis 304 each represent about 8.3 minutes of samples, for example. The heart rate data curve 306 was acquired from an actively engaged occupant in a simulator environment during manual and assisted driving.

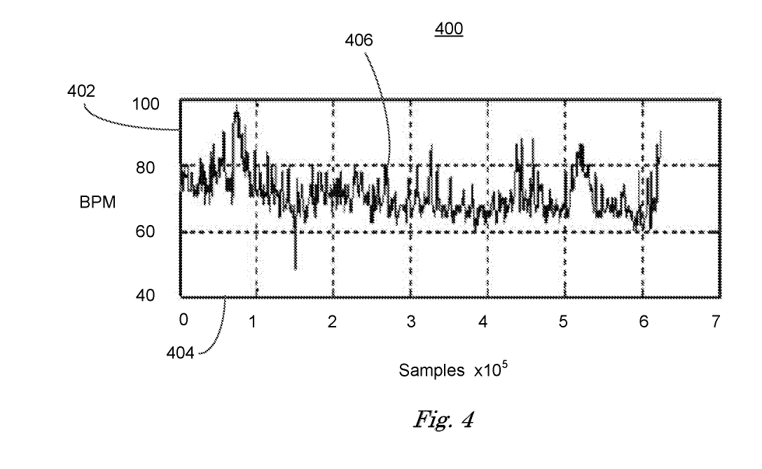

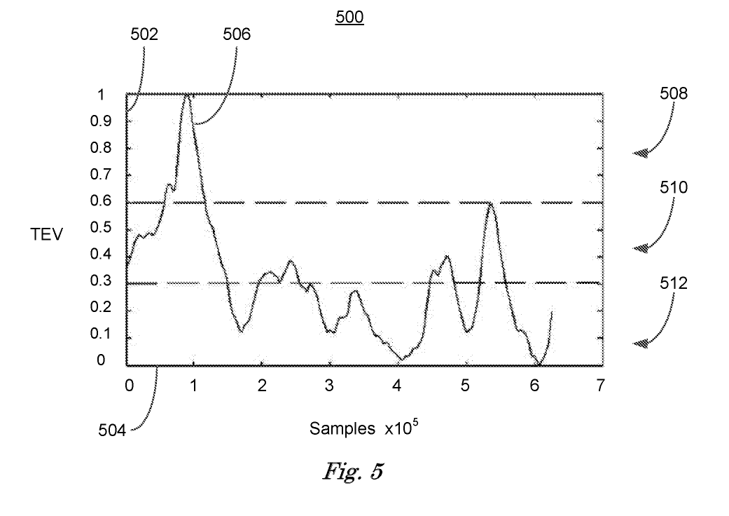

FIG. 4 is a graph of example heart rate data 400 from heart rate monitor 202 that graphs heart rate in beats per minute (BPM) on the Y-Axis 402 vs. number of samples×10⁵ on the X-Axis 404. FIG. 4 includes a heart rate data curve 406 acquired from an occupant in a simulator environment transitioning from engaged activity to low activity and to sleep. The engagement of the occupant transitions from engaged activity in the interval from sample “0” to about sample “1”, to low activity in the sample intervals from about sample “1” to about sample “3”, to sleep at about sample “3”, for example. Determining a transitional engagement value that identifies transitions in occupant's engagement may predict inattentive occupant behavior, as will be shown below in relation to FIG. 5.

Heart rate data 300 can be output to baseline computation and tracking process 204. Output means to transmit, transfer, send, write, or in any manner whatsoever output. The baseline computation and tracking process 204 acquires heart rate data and combines it with previously acquired heart rate data 300 to determine a baseline heart rate range. The baseline heart rate range can be expressed as a minimum heart rate Pₘᵢₙ and a heart rate range P_{range}.

The baseline range can be determined by acquiring a plurality of heart rate data 300 samples and determining the maximum and minimum values. Examination of the contextual data set will yield a sample minimum heart rate Iₘᵢₙ and sample heart rate range I_{range}. Baseline minimum heart rate Pₘᵢₙ and the heart rate range P_{range} can be updated to the sample minimum heart rate Iₘᵢₙ and sample heart rate range I_{range} for an individual.

Iₘᵢₙ and I_{range} may be obtained under various contexts to update Pₘᵢₙ and P_{range} as part of an individual learning process. For example, data may be obtained when the driver is piloting a vehicle, and during various assist states and categorized by context. “Context” means a level of vehicle human occupant (e.g., driver) activity in piloting the vehicle. A context is typically selected as a category of driver activity selected from a group of categories that describe the level of activity, such as “high activity piloting”, “low activity piloting”, “assisted piloting”, “not piloting”, “sleeping”, etc. In addition, heart rate data can be recorded from a wearable device prior to driving during a time the user may be sleeping may be used to obtain Iₘᵢₙ to update Pₘᵢₙ for the individual occupant. The value heart rate values to determine Iₘᵢₙ from the wearable device may be transmitted to the computing device 115. The number of control signals per unit time, e.g., per minute, for context to fall into a given category can be empirically determined, e.g., a driver having full control and fully alert can drive a vehicle in a test environment and/or on real roads and control signals can be recorded and used to establish context category thresholds for “high activity piloting.” Similar empirical data gathering could be performed for other categories.

When the occupant is actively driving for example, the context may be determined by computing device 115 by monitoring the control signals to controllers 112, 113, 114, and thereby determining the amount of piloting activity. Computing device 115 can count the number of control signals sent to controllers 112, 113, 114 based on inputs from occupant per unit time to determine if the driver is actively engaged in piloting thereby making the context equal to “high activity piloting” or “low activity piloting” depending upon the number of control signals received per unit time, for example. Context can be used by transition prediction system 200 to detect changes in occupant's activity level that can be used to adapt baseline minimum heart rate Pₘᵢₙ and the heart rate range P_{range} to activity levels representative of the context.

Returning to FIG. 2, heart rate monitor can also output the heart rate data 300, 400 to the Transitional Engagement Value (TEV) computation process 206. TEV is a measure of an occupant's attentiveness to piloting activities or virtual driver supervision. TEV computation process 206 determines a Transitional Engagement Value (TEV) based on the baseline range Pₘᵢₙ and P_{range} and a norm heart rate x̅ₖ. The norm heart rate in BPM at time k, can be calculated by the equation:

\hat{x}ₖ=α\hat{x}ₖ+(1−α)xₖ (1)

wherein the norm heart rate 4 is calculated by weighting the previous norm heart rate with a tunable constant α and adding it to the current heart rate xₖ weighted by 1−α. The tunable constant α is a value between 0 and 1 and may be chosen based on the desired time constant or response time to alert the occupant or advice the virtual driver. A typical value of a may be 0.97. For a faster response a lower value of a may be selected. For example, a may be relatively chosen as 0.85. A faster response may be required to alert the user during situational contexts including the time-of-day or traffic conditions.

TEV computation process 206 combines the norm heart rate 4 with baseline data Pₘᵢₙ and P_{range} to calculate the transitional engagement value at time k according to the equation:

$${TEV}_{k} = \frac{( {\overline{x}}_{k} - {P}_{min} )}{{P}_{range}} \qquad ( 2 )$$

where TEVₖ is the transitional engagement value at time k, and x̅ₖ, Pₘᵢₙ and P_{range} are calculated as above. Transitional engagement value can detect changes in an occupant's behavior towards piloting activity or virtual driver supervision and predict a transition in the occupant's engagement associated with inattentive behavior towards piloting activity. Inattention to piloting can be caused by drowsiness or sleep, for example.

FIG. 5 is a graph of transitional engagement 500, where TEVₖ, as calculated by equation (2), is plotted on the Y-Axis 502 vs. number of samples×10⁵ on the X-Axis 504. Each interval on the X-Axis 502 represents about 8.3 minutes of samples. TEV curve 506 is associated with acquired heart rate data 400 from an occupant in a simulator environment transitioning from engaged activity to low activity, and to sleep. In the sample interval below about “1”, TEV curve 506 is in the active region 508 where 0.6<TEV≤1.0. TEV is in the active region 508 indicates occupant's active, wakeful behavior towards piloting or virtual driver supervision at the time the sample was acquired.

In the sample interval between “1” and “2” the TEV curve 506 changes from active region 508 to transitional region 510, where 0.3<TEV≤0.6. TEV in the transitional region 510 indicates occupant's transition from active, wakeful behavior towards piloting to inattentive, sleepy behavior towards piloting or virtual driver supervision. Near sample “2”, TEV curve 506 begins entering sleepy region 512, where 0<TEV≤0.3 indicates occupant's inattentive, sleepy behavior towards piloting or virtual driver supervision.

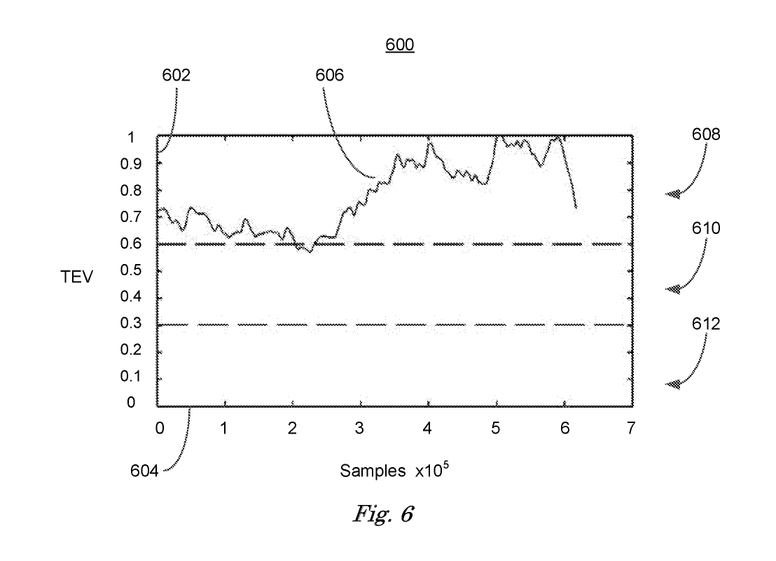

FIG. 6 is a graph of transitional engagement 600, where TEV, as calculated by equation (2), is plotted on the Y-Axis 602 vs. number of samples×10⁵ on the X-Axis 604. Each sample interval on the X-Axis represents about 8.3 minutes of samples. TEV curve 606 is associated with acquired heart rate data 300 from an occupant in a simulator environment during manual and assisted piloting. As can be seen, TEV curve 606 is, for the most part, in active region 608, only crossing into transition region 610 briefly and never approaching inattentive, sleepy region 612. During assisted driving the user was still relatively engaged physiologically and in the active region 608.

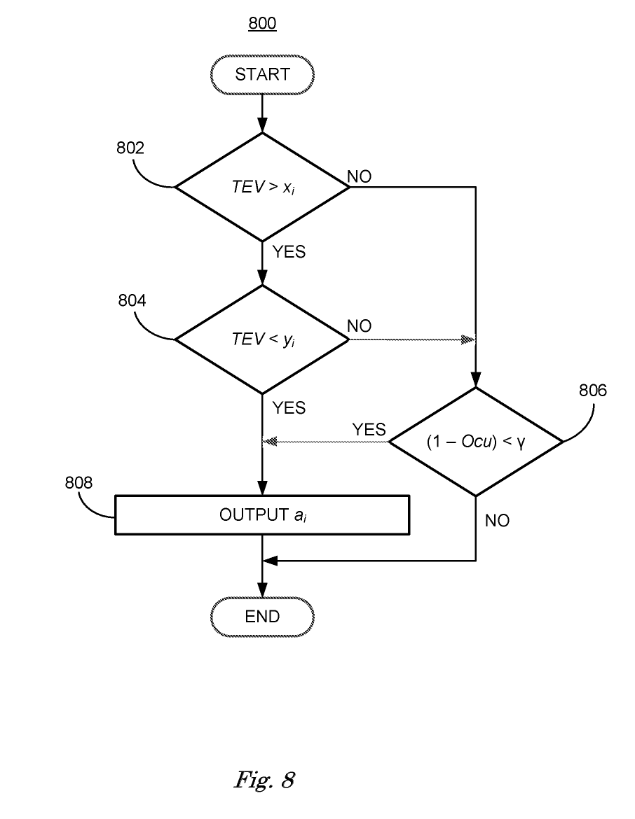

Returning to FIG. 2, comparative transition prediction system 200 can also include an eye motion monitor 208. Eye motion monitor can be a video-based sensor operative to acquire occupant's eye motion data. Eye motion data can be data that represents the location and direction of a vehicle occupant's gaze by locating the pupils of the occupant's eyes and determining their spatial orientation. Eye motion data can also represent the state of the occupant's eyelids, e.g. open, closed, blinking, etc. Eye motion data can be sampled and output to ocular behavior computation 210 on a periodic basis where occupant's eye motion can be processed to yield a variable Ocu that is proportional to eyelid closure. Ocu can assume values between 0 and 1 and is closer to 1 when eyelids are open and closer to 0 when eyelids are closed, for example. Ocular behavior computation 210 can output Ocu to decision computation 212 on a periodic basis. Decision computation 212 can input TEV from TEV computation 206 and Ocu from ocular behavior computation 210 and outputs signals including transition state aᵢ to alert occupant 216 and alert virtual driver 214 based on determining the occupant is in a transition state. FIG. 8 is a diagram of a flowchart, described in relation to FIGS. 1-6, of a process 800 for outputting transition state aᵢ. Process 800 can be implemented by a processor of computing device 115, taking as input information from sensors 116, and executing instructions and sending control signals via controllers 112, 113, 114, for example. Process 800 includes multiple steps taken in the disclosed order. Process 800 also includes implementations including fewer steps or can include the steps taken in different orders.

Process 800 depends upon predetermined values xᵢ, yᵢ, i and γ. Predetermined value i is an index from the set {0, 1, 2, 3} for example. i can be determined by an occupant preference or preset by the vehicle 110 manufacturer, for example. The value of i determines which of a set of predetermined values xᵢ, yᵢ, will be compared to the current TEV. Examples of predetermined values xᵢ, yᵢ include the values that separate active region 508, 608 from transition region 510, 610 and sleepy region 512, 612 in FIGS. 5 and 6.

Process 800 begins at step 802 where computing device 115 compares the current TEV with a predetermined value xᵢ. If TEV is greater than xᵢ, TEV is above the sleepy region 512, 612, for example and control passes to step 804, where TEV is compared with a predetermined value yᵢ. If TEV is less than yᵢ, TEV is below the active region 508, 608, for example and control passes to step 808. At step 808 process 800 has determined that TEV is above the sleepy region 512, 612 and below the active region 508, 608, and therefore TEV is in a transition region 510, 610 and occupant is therefore in a transition state.

The output from process 800 at step 808 depends upon the value of aᵢ. Table 1 includes example values of aᵢ for values of i={0, 1, 2, 3}.

TABLE 1
Transition state output values

| | |
|---|---|
| a₀ | no action |
| a₁ | signal alert occupant |
| a₂ | signal alert virtual driver |
| a₃ | signal alert occupant and alert virtual driver |

depending upon the predetermined value i, at step 808 computing device 115 can signal alert occupant 216, signal alert virtual driver 214, both, or neither.

At step 806 computing device 115 can compare (1−Ocu) with a predetermined value γ. A value of (1−Ocu) less than a predetermined value γ can indicate an eyelid closure rate that is associated with a transition state. A “YES” decision is an independent determination that occupant is in a transition state and inattentive behavior is predicted. If the decision at step 806 is “NO”, process 800 exits without outputting a transition state aᵢ.

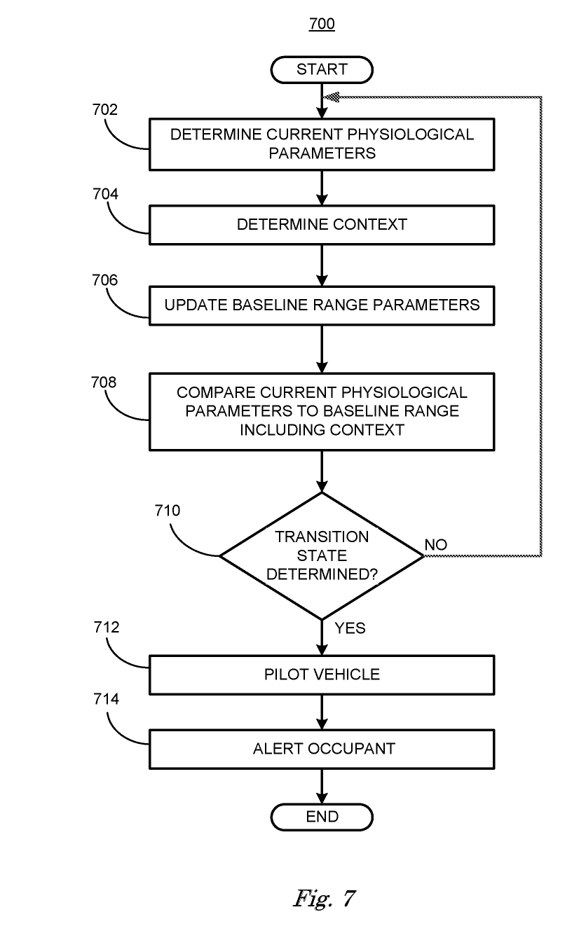

FIG. 7 is a diagram of a flowchart, described in relation to FIGS. 1-6, of a process 700 for piloting a vehicle by actuating one or more of a powertrain, brake, and steering in the vehicle upon determining a transition state. Process 700 can be implemented by a processor of computing device 115, taking as input information from sensors 116, and executing instructions and sending control signals via controllers 112, 113, 114, for example. Process 700 includes multiple steps taken in the disclosed order. Process 700 also includes implementations including fewer steps or can include the steps taken in different orders.

Process 700 starts at step 702 where computing device 115 determines current physiological parameters. Current physiological parameters include sampled heart rate data 300, and sampled eye motion data from eye motion monitor 208, as disclosed above in relation to FIG. 6. At step 704, computing device 115 determines a current context as discuss above in relation to FIG. 4. Current context represents the category of the current level of activity as determined by computing device 115 based on monitoring the current level of occupant piloting activity.

At step 706 computing device 115 updates the baseline range of physiological parameters by updating baseline range parameters Pₘᵢₙ and P_{range} as discussed above in relation to FIG. 2. In this fashion the baseline range parameters Pₘᵢₙ and P_{range} can be updated to correspond to the change in expected activity level.

At step 708 TEV computation process 206 of computing device 115 can determine TEV according to equation (2) and apply process 800 to determine transition state output aᵢ. At step 710, when process 800 outputs a transition state output aᵢ, at step 712 computing device 115 can control the vehicle without occupant intervention as discussed above in relation to FIG. 2 and at step 714 alert the occupant at discussed above in relation to FIG. 2.

At some point in time following determination of a transition state output aᵢ, the occupant's TEV can rise to an active, wakeful level, e.g., the occupant has been awakened by the alert. Determination of an active, wakeful TEV for some number of samples and possibly an action by the occupant such as entering a code on a keypad, for example, could be required to return piloting control to the occupant.

In summary, process 700 is a process that can acquire physiological parameters from an occupant, determine the context, update baseline parameter range and compare the physiological parameters to the baseline range based on the context to determine a transition state output aᵢ. Depending upon predetermined values, transition state output aᵢ can include sending signals to alert occupant 216 and alert virtual driver 214 whereupon computing device 115 can alert the occupant and pilot vehicle 110 autonomously for some period of time.

Computing devices such as those discussed herein generally each include instructions executable by one or more computing devices such as those identified above, and for carrying out blocks or steps of processes described above. For example, process blocks discussed above may be embodied as computer-executable instructions.

Computer-executable instructions may be compiled or interpreted from computer programs created using a variety of programming languages and/or technologies, including, without limitation, and either alone or in combination, Java™, C, C++, Visual Basic, Java Script, Perl, HTML, etc. In general, a processor (e.g., a microprocessor) receives instructions, e.g., from a memory, a computer-readable medium, etc., and executes these instructions, thereby performing one or more processes, including one or more of the processes described herein. Such instructions and other data may be stored in files and transmitted using a variety of computer-readable media. A file in a computing device is generally a collection of data stored on a computer readable medium, such as a storage medium, a random access memory, etc.

A computer-readable medium includes any medium that participates in providing data (e.g., instructions), which may be read by a computer. Such a medium may take many forms, including, but not limited to, non-volatile media, volatile media, etc. Non-volatile media include, for example, optical or magnetic disks and other persistent memory. Volatile media include dynamic random access memory (DRAM), which typically constitutes a main memory. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, or any other medium from which a computer can read.

All terms used in the claims are intended to be given their plain and ordinary meanings as understood by those skilled in the art unless an explicit indication to the contrary in made herein. In particular, use of the singular articles such as “a,” “the,” “said,” etc. should be read to recite one or more of the indicated elements unless a claim recites an explicit limitation to the contrary.

The term “exemplary” is used herein in the sense of signifying an example, e.g., a reference to an “exemplary widget” should be read as simply referring to an example of a widget.

The adverb “approximately” modifying a value or result means that a shape, structure, measurement, value, determination, calculation, etc. may deviate from an exact described geometry, distance, measurement, value, determination, calculation, etc., because of imperfections in materials, machining, manufacturing, sensor measurements, computations, processing time, communications time, etc.

In the drawings, the same reference numbers indicate the same elements. Further, some or all of these elements could be changed. With regard to the media, processes, systems, methods, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain embodiments, and should in no way be construed so as to limit the claimed invention.

We claim:

1. A method, comprising:
determine a level of activity by an occupant piloting a vehicle and assigning a category based on the determined level of activity;
determining a baseline range of one or more physiological parameters by updating the baseline range of physiological parameters based on the determined level of activity;
determining one or more current physiological parameters for the occupant;
determining the occupant is in a transition state which indicates a transition to inattentive behavior by comparing the current physiological parameters to the baseline range of physiological parameters including determining a norm of the current physiological parameters according to the determined level of activity; and
actuating one or more of an alert, a powertrain, brake, and steering in the vehicle upon determining the transition state.

2. The method of claim 1, wherein the determined level of activity includes a level and duration of piloting activity by the occupant.

3. The method of claim 1, wherein updating the baseline range of physiological parameters includes periodically acquiring physiological parameters and the determined level of activity from the occupant and therewith adapting the baseline range of physiological parameters.

4. The method of claim 1, further comprising:
piloting the vehicle autonomously when the transition state is determined.

5. The method of claim 1, further comprising:
determining the baseline range of physiological parameters and one or more current physiological parameters for the occupant includes acquiring physiological signals from the occupant with a wearable device.

6. The method of claim 5, wherein the physiological signals include heart rate.

7. The method of claim 1, further comprising:
determining the baseline range of physiological parameters and one or more current physiological parameters for the occupant includes acquiring physiological signals from the occupant with a non-contact device.

8. The method of claim 7, wherein the physiological signals include eye motion.

9. An apparatus, comprising:
a processor;
a memory, the memory storing instructions executable by the processor to:
determine a level of activity by an occupant piloting a vehicle and assigning a category based on the determined level of activity;
determine a baseline range of one or more physiological parameters by updating the baseline range of physiological parameters based on the determined level of activity;
determine one or more current physiological parameters for the occupant;
determine the occupant is in a transition state which indicates a transition to inattentive behavior by comparing the current physiological parameters to the baseline range of physiological parameters including determining a norm of the current physiological parameters according to the determined level of activity; and
actuate one or more of an alert, a powertrain, brake, and steering in the vehicle upon determining the transition state.

10. The apparatus of claim 9, wherein the determined level of activity includes a level and duration of piloting activity by the occupant.

11. The apparatus of claim 9, wherein updating the baseline range of physiological parameters includes periodically acquiring physiological parameters and the determined level of activity from the occupant and therewith adapting the baseline range of physiological parameters.

12. The apparatus of claim 9, further comprising:
pilot the vehicle autonomously upon determining the transition state.

13. The apparatus of claim 9, further comprising:
determine the baseline range of physiological parameters and one or more current physiological parameters for the occupant includes acquire physiological signals from the occupant with a wearable device.

14. The apparatus of claim 13, wherein the physiological signals include heart rate.

15. The apparatus of claim 9, further comprising:
determining the baseline range of physiological parameters and one or more current physiological parameters for the occupant includes acquire physiological signals from the occupant with a non-contact device.

16. The apparatus of claim 15, wherein the physiological signals include eye motion.

17. A vehicle, comprising:
a processor;
a memory, the memory storing instructions executable by the processor to:
determine a level of activity by an occupant piloting the vehicle and assigning a category based on the determined level of activity;
determine a baseline range of one or more physiological parameters by updating the baseline range of physiological parameters based on the determined level of activity;
determine one or more current physiological parameters for the occupant;
determine the occupant is in a transition state which indicates a transition to inattentive behavior by comparing the current physiological parameters to the baseline range of physiological parameters including determining a norm of the current physiological parameters according to the determined level of activity; and
actuate one or more of an alert, a powertrain, brake, and steering in the vehicle upon determining the transition state.

18. The vehicle of claim 17, wherein the determined level of activity includes a level and duration of piloting activity by the occupant.

19. The vehicle of claim 18, wherein updating the baseline range of physiological parameters includes periodically acquiring physiological parameters and the determined level of activity from the occupant and therewith adapting the baseline range of physiological parameters.

20. The vehicle of claim 17, further comprising:
pilot the vehicle autonomously upon determining the transition state.
